(19) United States
(12) Patent Application Publication
(10) Pub. No.: US 2005/0021118 A1
(43) Pub. Date: Jan. 27, 2005

(54) Apparatuses and systems for applying electrical stimulation to a patient

(76) Inventors: Chris Genau, Seattle, WA (US); Kent Leyde, Redmond, WA (US); Jay Miazga, Seattle, WA (US); Corinne J. Stern, Kent, WA (US)

Correspondence Address: PERKINS COIE LLP;PATENT-SEA, P.O. BOX 1247, SEATTLE, WA, 98111-1247, US

(21) Appl. No.: 10/877,830

(22) Filed: Jun. 25, 2004

Related U.S. Application Data
(63) Continuation-in-part of application No. 10/260,227, filed on Sep. 27, 2002.
(63) Continuation-in-part of application No. 09/802,808, filed on Mar. 8, 2001.
(60) Provisional application No. 60/482,937, filed on Jun. 26, 2003.
(60) Provisional application No. 60/325,978, filed on Sep. 28, 2001.
(60) Provisional application No. 60/217,981, filed on Jul. 13, 2000.

(51) Int. Cl. A61N 1/05

(52) U.S. Cl. 607/116

(57) ABSTRACT

Apparatuses and systems for applying electrical stimulation to a site on a patient. In one embodiment, an implantable electrode assembly includes an electrode array carried by a flexible support member. The electrode array can include a first plurality of electrodes spaced apart from a second plurality of electrodes. The first plurality of electrodes can be connected to a first lead line, and the second plurality of electrodes can be similarly connected to a second lead line. The first and second lead lines can be housed in a cable extending away from the support member. A distal end of the cable can include a connector for coupling the lead lines to an implantable pulse generator or other stimulus unit. In operation, the stimulus unit can bias the first plurality of electrodes at a first potential and the second plurality of electrodes at a second potential to generate an electric field proximate to a stimulation site.

### CROSS-REFERENCE TO RELATED APPLICATIONS INCORPORATED BY REFERENCE

This application claims the benefit of copending U.S. Provisional Patent Application No. 60/482,937, filed Jun. 26, 2003, and is a continuation-in-part of U.S. patent application Ser. No. 10/260,227, filed Sep. 27, 2002, which claims the benefit of U.S. Provisional Patent Application No. 60/325,978, filed Sep. 28, 2001, and which is a continuation-in-part of U.S. patent application Ser. No. 09/802,808, filed Mar. 8, 2001, which claims the benefit of U.S. Provisional Patent Application No. 60/217,981, filed Jul. 31, 2000.

U.S. patent application Ser. Nos. 10/260,227, 09/802,808, 10/260,720, and 10/112,301; and U.S. Provisional Patent Application Nos. 60/482,937, 60/325,978, and 60/217,981; are incorporated into the present disclosure in their entireties by reference.

### TECHNICAL FIELD

The following disclosure is related to apparatuses and systems for applying neural stimulation to a patient, for example, at a surface site on the patient's cortex.

### BACKGROUND

A wide variety of mental and physical processes are controlled or influenced by neural activity in particular regions of the brain. The neural functions in some areas of the brain (e.g., the sensory or motor cortices) are organized according to physical or cognitive functions. Several other areas of the brain also appear to have distinct functions in most individuals. In the majority of people, for example, the occipital lobes relate to vision, the left interior frontal lobes relate to language, and the cerebral cortex appears to be involved with conscious awareness, memory, and intellect.

Many problems or abnormalities can be caused by damage, disease, and/or disorders of the brain. Effectively treating such abnormalities may be very difficult. For example, a stroke is a common condition that damages the brain. Strokes are generally caused by emboli (i.e., obstruction of a blood vessel), hemorrhages (i.e., rupture of a blood vessel), or thrombi (i.e., clotting) in the vascular system of a specific region of the brain. Such events generally result in a loss or impairment of neural function (e.g., neural functions related to facial muscles, limbs, speech, etc.). Stroke patients are typically treated using various forms of physical therapy that rehabilitate the loss of function of a limb or another affected body part. Stroke patients may also be treated using physical therapy plus an adjunctive therapy such as amphetamine treatment. For most patients, however, such treatments are minimally effective and little can be done to improve the function of an affected body part beyond the recovery that occurs naturally without intervention.

Problems or abnormalities in the brain are often related to electrical and/or chemical activity in the brain. Neural activity is governed by electrical impulses or “action potentials” generated in neurons and propagated along synaptically connected neurons. When a neuron is in a quiescent state, it is polarized negatively and exhibits a resting membrane potential typically between −70 and −60 mV. Through chemical connections known as synapses, any given neuron receives excitatory and inhibitory input signals or stimuli from other neurons. A neuron integrates the excitatory and inhibitory input signals it receives and generates or fires a series of action potentials when the integration exceeds a threshold potential. A neural firing threshold, for example, may be approximately −55 mV.

It follows that neural activity in the brain can be influenced by electrical energy supplied from an external source such as a waveform generator. Various neural functions can be promoted or disrupted by applying an electrical current to the cortex or other region of the brain. As a result, researchers have attempted to treat physical damage, disease, and disorders in the brain using electrical or magnetic stimulation signals to control or affect brain functions.

Transcranial electrical stimulation (TES) is one such approach that involves placing an electrode on the exterior of the scalp and delivering an electrical current to the brain through the scalp and skull. Another treatment approach, transcranial magnetic stimulation (TMS), involves producing a magnetic field adjacent to the exterior of the scalp over an area of the cortex. Yet another treatment approach involves direct electrical stimulation of neural tissue using implanted electrodes.

The neural stimulation signals used by these approaches may comprise a series of electrical or magnetic pulses that can affect neurons within a target neural population. Stimulation signals may be defined or described in accordance with stimulation signal parameters that include pulse amplitude, pulse frequency, duty cycle, stimulation signal duration, and/or other parameters. Electrical or magnetic stimulation signals applied to a population of neurons can depolarize neurons within the population toward their threshold potentials. Depending upon stimulation signal parameters, this depolarization can cause neurons to generate or fire action potentials.

Neural stimulation that elicits or induces action potentials in a functionally significant proportion of the neural population to which the stimulation is applied is referred to as supra-threshold stimulation; neural stimulation that fails to elicit action potentials in a functionally significant proportion of the neural population is defined as sub-threshold stimulation. In general, supra-threshold stimulation of a neural population triggers or activates one or more functions associated with the neural population, but sub-threshold stimulation by itself does not trigger or activate such functions. Supra-threshold neural stimulation can induce various types of measurable or monitorable responses in a patient. For example, supra-threshold stimulation applied to a patient's motor cortex can induce muscle fiber contractions in an associated part of the body to produce an intended type of therapeutic, rehabilitative, or restorative result.

FIG. 1 is a top isometric view of an implantable electrode assembly 100 configured in accordance with the prior art. The prior art electrode assembly 100 can be at least generally similar in structure and function to the Resume II electrode assembly provided by Medtronic, Inc., of 710 Medtronic Parkway, Minneapolis, Minn. 55432-5604. The electrode assembly 100 is typically used to deliver electrical stimulation to a spinal cord site of a patient and includes a plurality of plate electrodes 104a-d carried by a flexible substrate 102. A polyester mesh 110 can be molded into the substrate 102 to increase the tensile strength of the substrate 102. A cable 106 houses four individually insulated leads 108a-d that extend into the substrate 102. After entering the substrate 102, the first lead 108a is separated from the other leads and crimped to the top of the first electrode 104a. The remaining leads 108b, 108c, and 108d are similarly separated and crimped to the tops of the remaining electrodes 104b, 104c, and 104d, respectively. A distal end of the cable 106 includes an in-line connector 112 configured to be received by a receptacle 114. Joining the connector 112 to the receptacle 114 forms an intermediate coupling between the electrode assembly 100 and a power source (not shown) configured to provide electrical pulses to one or more of the electrodes 104. The receptacle 114 includes four set-screws 115a-d configured to individually engage corresponding contacts 113a-d on the connector 112 when the connector 112 is inserted into the receptacle 114. Each of the contacts 113a-d is individually connected to a corresponding one of the leads 108a-d. As a result, proper joining of the connector 112 to the receptacle 114 allows the power source to apply a different electrical potential to each of the electrodes 104a-d.

One shortcoming of the prior art electrode assembly 100 is that the substrate 102 has a thickness 101 of about 2.5 mm. Although this thickness may be acceptable for certain spinal cord applications, it can present problems in intracranial applications where space between the skull and cortex is limited. For example, one such problem is that implantation of the electrode assembly 100 in the narrow confines between the skull and cortex can cause the electrode assembly 100 to apply localized pressure to the cortex of the patient.

Another shortcoming of the electrode assembly 100 is associated with the intermediate coupling between the connector 112 and the receptacle 114. This coupling is relatively large and, accordingly, it may be difficult to push through a tunnel extending, for example, from a subclavicular region, along the back of the neck, and around the skull of a patient. Not only is this coupling relatively large, but it is also relatively fragile and prone to damage during use. Such damage can include breakage of the connector 112 due to over-tightening of the corresponding set-screws 115. In addition, use of an intermediate coupling can increase the risk of fatigue failure of the lead as it is bent around the relatively sharp radius of the receptacle 114.

A further shortcoming associated with the prior art electrode assembly 100 is the relatively time-intensive manufacturing process required to break out each individually insulated lead 108 from the cable 106 and then crimp each individual lead 108 to its corresponding electrode 104. In addition, these crimps may be prone to breakage from flexing of the substrate 102 during implantation, which renders the electrode assembly 100 at least partially inoperative. If inoperative, the electrode assembly 100 may have to be removed from the patient, and a second invasive procedure may be necessary to implant another fully operative electrode assembly.

In spinal cord therapy, it is often desirable to focus electrical stimulation within 1-2 mm of a target location to enhance the efficacy of the procedure. It is for this reason that the electrode assembly 100 includes a quadripolar array of electrodes 104 providing multiple stimulation combinations within a relatively short distance. The quadripolar array allows the relative electrical potentials between any two electrodes to be tuned to focus the electrical stimulation in the narrow space between the two electrodes. While this configuration may be useful in certain spinal cord applications, it may be less useful in those applications where broader coverage is desired. Such applications may include, for example, certain applications where broader stimulation of the cortical site is desired.

### BRIEF DESCRIPTION OF THE DRAWINGS

FIG. 1 is a top isometric view of an implantable electrode assembly configured in accordance with the prior art.

FIG. 2 is a top, partially hidden isometric view of an implantable electrode assembly configured in accordance with an embodiment of the invention.

FIG. 3A is an exploded top isometric view of the electrode assembly of FIG. 2 configured in accordance with an embodiment of the invention.

FIG. 3B is a top isometric view of the electrode assembly of FIG. 2 in a partially assembled state with a portion of a support member omitted for clarity.

FIG. 4 is a top isometric view of a partially assembled electrode assembly configured in accordance with another embodiment of the invention.

FIG. 5A is an exploded top isometric view of an implantable electrode assembly configured in accordance with a further embodiment of the invention.

FIG. 5B is an enlarged, partial cutaway isometric view of a plurality of interconnected electrodes from the electrode assembly of FIG. 5A.

FIG. 6 is a partially exploded top isometric view of an electrode assembly configured in accordance with another embodiment of the invention.

FIG. 7 is an enlarged, cutaway isometric view of a portion of an electrode assembly having a cable configured in accordance with an embodiment of the invention.

FIG. 8 is a side view illustrating a system for applying electrical stimulation to a surface on the cortex of a patient in accordance with an embodiment of the invention.

FIG. 9 is an enlarged cross-sectional view of an electrode assembly implanted at a stimulation site on a patient in accordance with an embodiment of the invention.

FIG. 10 is an enlarged, cross-sectional side view of the electrode assembly of FIG. 6 being installed at a stimulation site in accordance with an embodiment of the invention.

FIG. 11 is a top, partially hidden isometric view of an electrode assembly configured in accordance with another embodiment of the invention.

FIG. 12 is a partially exploded top isometric view of an electrode assembly configured in accordance with yet another embodiment of the invention.

### DETAILED DESCRIPTION

The present disclosure describes apparatuses and systems for applying electrical stimulation to cortical and other sites on a patient, and associated methods of manufacturing such apparatuses. Stimulation systems and methods described herein may be used to treat a variety of neurological conditions. Depending on the nature of a particular condition, neural stimulation applied or delivered in accordance with various embodiments of such systems and/or methods may facilitate or effectuate reorganization of interconnections or synapses between neurons to (a) provide at least some degree of recovery of a lost function; and/or (b) develop one or more compensatory mechanisms to at least partially overcome a functional deficit. Such reorganization of neural interconnections may be achieved, at least in part, by a change in the strength of synaptic connections through a process that corresponds to a mechanism commonly known as Long-Term Potentiation (LTP). Electrical stimulation applied to one or more target neural populations either alone or in conjunction with behavioral activities and/or adjunctive or synergistic therapies may facilitate or effectuate neural plasticity and the reorganization of synaptic interconnections between neurons.

One embodiment of a system for applying electrical stimulation to a cortical stimulation site in accordance with the invention includes an implantable electrode assembly connected to a stimulus unit. The stimulus unit can be an implantable pulse generator (IPG) having at least a first terminal that can be biased at a first electrical potential and a second terminal that can be biased at a second electrical potential. The implantable electrode assembly can include an array of electrodes carried by a flexible support member configured to be placed at the stimulation site. A first conductor or lead can connect a first plurality of the electrodes to the first terminal of the IPG, and a second conductor or lead can connect a second plurality of the electrodes to the second terminal of the IPG. In operation, the IPG can bias the first plurality of electrodes at the first potential and the second plurality of electrodes at the second potential to generate an electric field at least proximate to the stimulation site for promoting neuroplasticity. As used herein, the term “stimulation site” refers to a location where target neurons for a particular therapy are located. For example, in certain embodiments, such locations may be proximate to the cortex, either on the dura mater or beneath the dura mater.

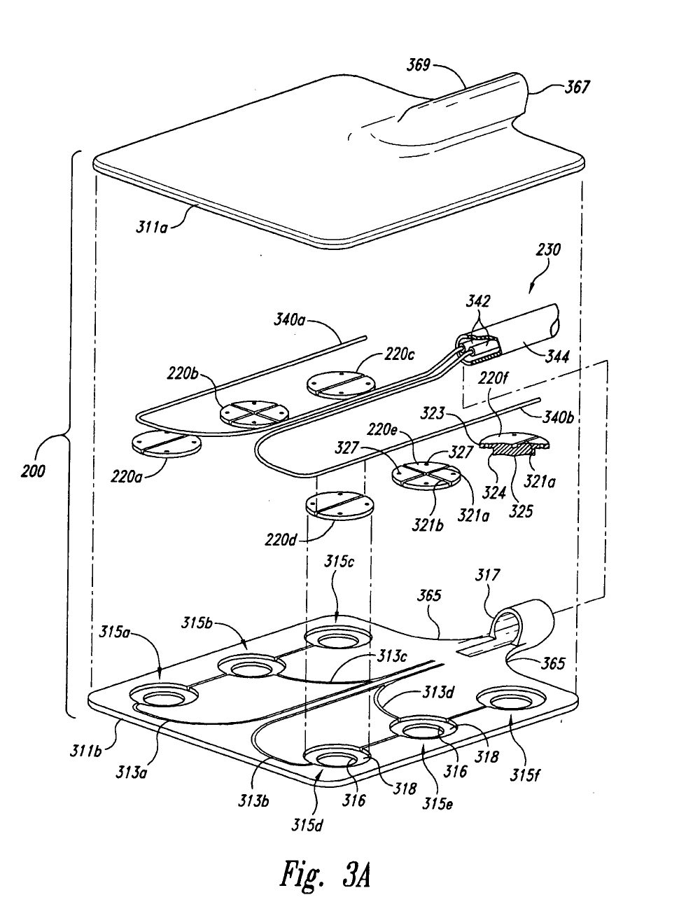

Certain specific details are set forth in the following description and in FIGS. 2-11 to provide a thorough understanding of various embodiments of the invention. Other details describing structures and systems well known to those of ordinary skill in the relevant art are not set forth in the following description, however, to avoid unnecessarily obscuring the description of various embodiments of the invention. Dimensions, angles, and other specifications shown in the following figures are merely illustrative of particular embodiments of the invention. Accordingly, other embodiments can have other dimensions, angles, and specifications without departing from the spirit or scope of the invention. In addition, still other embodiments of the invention can be practiced without several of the details described below.

In the figures, identical reference numbers identify identical or at least generally similar elements. To facilitate the discussion of any particular element, the most significant digit or digits of any reference number refer to the figure in which that element is first introduced. For example, element 210 is first introduced and discussed with reference to FIG. 2.

FIG. 2 is a top partially hidden isometric view of an implantable electrode assembly 200 configured in accordance with an embodiment of the invention. In one aspect of this embodiment, the electrode assembly 200 includes an electrode array comprising a first plurality of electrodes 221 (illustrated as electrodes 220a-c) and a second plurality of electrodes 222 (illustrated as electrodes 220d-f). The electrodes 220 can be carried by a flexible support member 210 configured to place each electrode 220 in contact with a stimulation site of a patient when the support member 210 is placed at the stimulation site. The electrodes 220 are connected to conductors or lead lines (not shown in FIG. 2) housed in a cable 230. A distal end of the cable 230 can include a connector 233 for connecting the lead lines to an IPG or other stimulation unit for electrical biasing of the electrodes 220. In operation, the first plurality of electrodes 221 can be biased at a first potential and the second plurality of electrodes 222 can be biased at a second potential at any given time. The different potentials can generate electrical pulses in the patient at, or at least proximate to, the stimulation site. In a different embodiment, all of the electrodes can be at the same potential for an isopolar stimulation process. These electric pulses may provide or induce an intended therapeutic result in the patient, for example, through neuroplasticity and the reorganization of synaptic interconnections between neurons.

Although the electrode assembly 200 of the illustrated embodiment includes a 2×3 electrode array (i.e., 2 rows of 3 electrodes each), in other embodiments, electrode assemblies in accordance with the present invention can include more or fewer electrodes in other types of symmetrical and asymmetrical arrays. For example, in one other embodiment, such an electrode assembly can include a 1×2 electrode array. In another embodiment, such an electrode assembly can include a 2×5 electrode array. In a further embodiment, such an electrode assembly can include a single electrode for isopolar stimulation. Furthermore, although the electrodes 220 appear to be evenly spaced along respective sides of the electrode assembly 200, in other embodiments, the electrodes 220 can have other spacing. For example, in one other embodiment, the space between the first electrode 220a and the second electrode 220b can be different than the space between the second electrode 220b and the third electrode 220c. Similarly, in this embodiment, the space between the fourth electrode 220d and the fifth electrode 220e can be different than the space between the fifth electrode 220e and the sixth electrode 220f. Several other electrode configurations are shown and described in U.S. application Ser. No. 10/112,301, filed Mar. 28, 2002, which is herein incorporated in its entirety by reference. Accordingly, aspects of the electrode assemblies disclosed herein in accordance with the present invention are not limited to the embodiments illustrated, but instead they can be applied to other electrode assemblies having other configurations.

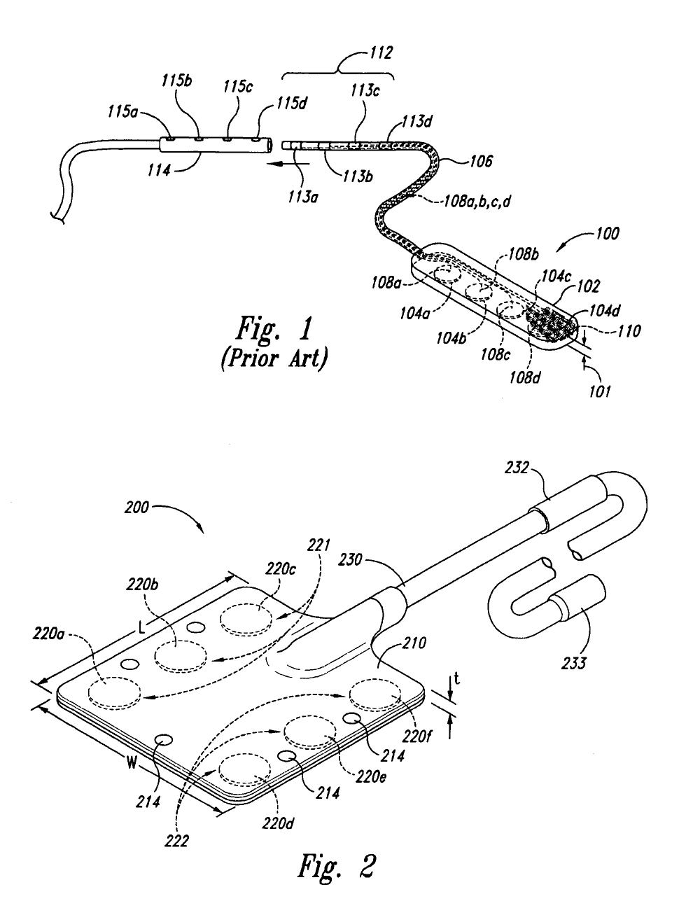

In another aspect of this embodiment, the electrode assembly 200 can be shaped and sized to facilitate intracranial use without installation difficulties or patient discomfort. For example, in one embodiment, the support member 210 can have a relatively thin thickness T of about 1.25 mm. This thickness is less likely to apply localized pressure to the cortex of the patient than thicker devices, such as the prior art electrode assembly 100 of FIG. 1 that has a thickness of about 2.5 mm. In other embodiments, the support member 210 can have other thicknesses. For example, in one other embodiment, the electrode assembly 200 can have a thickness of about 1.5 mm or greater. In another embodiment, the electrode assembly 200 can have a thickness T of about 1 mm or less. In a further aspect of this embodiment, the electrode assembly 200 can have a length L of about 27 mm, and a width W of about 26 mm. In other embodiments, the electrode assembly 200 can have other shapes and different dimensions, depending on factors such as the size of the individual electrodes 220 and/or the size and arrangement of the corresponding electrode array.

In yet another aspect of this embodiment, the electrode assembly 200 can include one or more coupling apertures 214 extending through the periphery of the support member 210. As explained in greater detail below, in one embodiment, the coupling apertures 214 can facilitate temporary attachment of the electrode assembly 200 to dura mater at, or at least proximate to, a stimulation site. The electrode assembly 200 can also include a protective sleeve 232 disposed over a portion of the cable 230. In one embodiment, the sleeve 232 can be manufactured from a silicone material having a relatively high durometer. In other embodiments, other suitable materials can be used to protect the cable 230 from abrasion and provide strain relief for the support member 210. As further explained below, in one embodiment, the sleeve 232 can protect the cable 230 from abrasion resulting from contact with the edge of an access hole formed in the patient's skull.

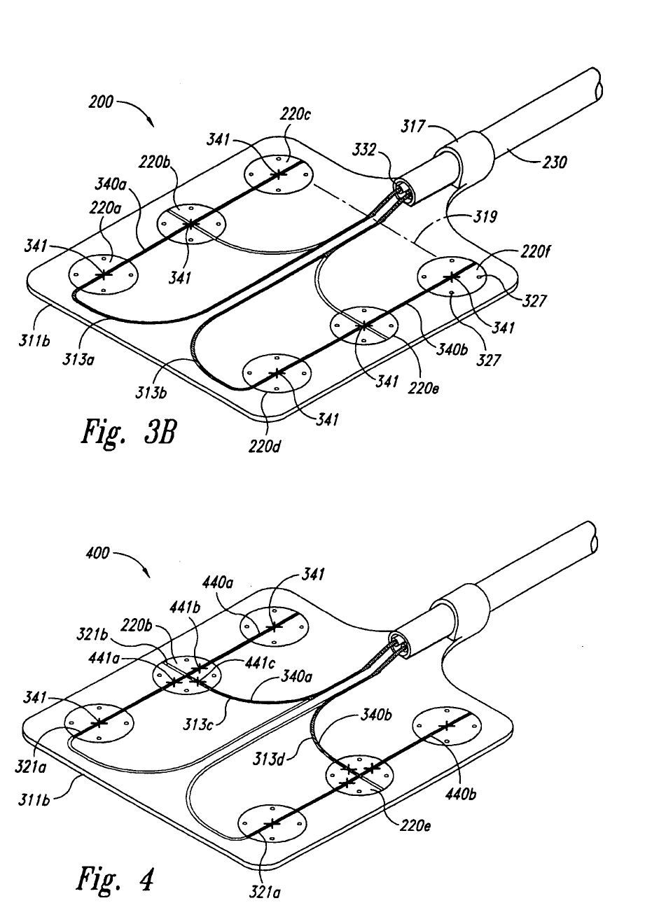

FIG. 3A is an exploded top isometric view of the electrode assembly 200 of FIG. 2 in accordance with an embodiment of the invention. FIG. 3B is a corresponding isometric view of the electrode assembly 200 in a partially assembled state with a top portion of the support member 210 omitted for clarity. Referring first to FIG. 3A, and specifically to the electrode 220f that is partially cut away for purposes of illustration, one aspect of this embodiment is that each of the electrodes 220 includes a first shoulder portion 323 and a second base portion 324 extending downwardly from the shoulder portion 323. The base portion 324 can include a contact surface 325 that is at least generally flat and configured to contact a tissue surface when positioned at a stimulation site. Each of the electrodes 220 can further include at least a first groove 321a extending through the shoulder portion 323. Some of the electrodes 220 (e.g., the electrodes 220b and 220e) can also include a second groove 321b extending through the shoulder portion 323 and crossing the first groove 321a.

In addition to the grooves 321, in one embodiment, each of the electrodes 220 can also include a plurality of adhesive apertures 327 extending axially through the shoulder portions of the electrodes 220. As explained below with reference to FIG. 3B, the adhesive apertures 327 may facilitate bonding of the electrodes 220 to the support member 210.

The electrodes 220 may be comprised of various electrically conductive materials. For example, in one embodiment, the electrodes 220 can include platinum and iridium in about a 9-to-1 ratio, respectively. In other embodiments, the electrodes 220 can include platinum and iridium in other ratios. In a further embodiment, the electrodes 220 can include only platinum. In yet other embodiments, the electrodes 220 can include other conductive materials suitable for patient implantation in medical applications such as stainless steel, nickel, titanium and/or gold. In still further embodiments, the electrodes 220 can include material coatings to increase the effective surface area of the electrodes 220 and/or decrease the electrical impedance at the tissue interface. Such coatings can include iridium, titanium oxide films, and/or metal blacks.

The electrodes 220 can be manufactured using a number of different methods in various embodiments. For example, in one embodiment, the electrodes 220 can be machined from stock. In another embodiment, the electrodes 220 can be cast. In a further embodiment, the electrodes 220 can be forged. In yet another embodiment, the electrodes 220 can be stamped from a thin sheet of material to provide the necessary cross-sectional shape. In still further embodiments, it is expected that still other methods can be used to manufacture the electrodes 220.

Although the electrodes 220 of the illustrated embodiment are at least generally round, in other embodiments, the electrodes 220 can have other geometrical shapes. For example, in one other embodiment, the electrodes 220 can be at least generally square or have other rectangular shapes. In further embodiments, the electrodes 220 can have other multi-sided shapes, such as triangles, octagons or hexagons. In yet other embodiments, the electrodes can have oval or elliptical shapes. In still further embodiments, it is expected that electrodes can have still other shapes, such as irregular shapes, depending on the particular application.

In another aspect of this embodiment, the grooves 321 in the electrodes 220 are configured to receive conductors or lead lines 340 (illustrated as a first lead line 340a and a second lead line 340b). In the illustrated embodiment, for example, the first grooves 321a in the first plurality of electrodes 221 receive a distal portion of the first lead line 340a, and the first grooves 321a in the second plurality of electrodes 222 similarly receive a distal portion of the second lead line 340b. Recessing the lead lines 340 in the grooves 321 can favorably reduce the overall thickness of the electrode assembly 200 as compared to, for example, extending the lead lines 340 over the tops of the electrodes 220 for attachment by crimping or some other method. As described in greater detail below, the lead lines 340 can be connected to a stimulus unit to produce a desired electric field between the first plurality of electrodes 221 and the second plurality of electrodes 222.

The lead lines 340 may be comprised of various electrically conductive materials. In one embodiment, for example, the lead lines 340 can include MP35N quadrifiler coil wire having a 0.254 mm outside diameter. Such coil wire may be provided by Lake Region Manufacturing, VNS-001-01K. In other embodiments, the lead lines 340 can include other types of electrically conductive wire. For example, in one other embodiment, the lead lines 340 can include single-strand MP35N wire. In yet another embodiment, the lead lines 340 can include multi-strand MP35N wire, such as 21-strand MP35N wire. Multi-strand wire may have certain advantages over other types of wire in selected embodiments. For example, multi-strand wire may cost less than coil wire, may have a higher tensile strength, and may have a lower impedance. In addition to the forgoing materials, the lead lines 340 can also include drawn filled tubing (DFT) materials, such as those provided by Fort Wayne Metals of 9609 Indianapolis Road, Fort Wayne, Ind. 46809. Such DFT wire materials can include various outer tube/core combinations. For example, the outer tube materials can include MP35N, 316LVM, Nitinol, Conichrome, and titanium alloys, among others; and the core materials can include gold, silver, platinum and tungsten, among others.

In a further aspect of this embodiment, the support member 210 includes a top or first portion 311a and a complimentary bottom or second portion 311b. The second portion 311b can include a plurality of electrode ports 315a-f configured to receive the electrodes 220a-f, respectively. In the illustrated embodiment, each electrode port 315 includes a contact aperture 316 and an annular recess 318 formed concentrically around the contact aperture 316. Each of the contact apertures 316 is configured to receive the base portion 324 of a corresponding electrode 220. Similarly, each of the annular recesses 318 is configured to receive at least part of the shoulder portion 323 of the corresponding electrode 220. In this manner, at least a portion of the contact surface 325 of each electrode 220 is exposed through the contact aperture 316 when the electrode 220 is fully installed in the electrode port 315. This positioning allows each electrode 220 to contact a tissue surface when the support member 210 is placed at a stimulation site.

In yet another aspect of this embodiment, the second portion 311b of the support member 210 can include a plurality of preformed grooves 313 (shown as a first groove 313a, second groove 313b, a third groove 313c, and a fourth groove 313d). The grooves 313 can extend from one or more of the electrode ports 315 to at least proximate a collar 317. The grooves 313 are configured to receive exposed portions of the lead lines 340 extending between the electrodes 220 and the cable 230. For example, in the illustrated embodiment, the first groove 313a receives an exposed portion of the first lead line 340a, and the second groove 313b receives an exposed portion of the second lead line 340b. The curved paths formed by the grooves 313 between the electrodes 220 and the cable 230 are shaped and sized to reduce strain between the lead lines 340 and the electrodes 220 when the support member 210 is flexed, stretched, or otherwise manipulated during use. This feature can reduce the likelihood of breaking a connection between one of the lead lines 340 and one of the electrodes 220 during implantation of the electrode assembly 200. In one embodiment, the grooves 313 can have a generally U-shaped cross-section. In another embodiment, the grooves 313 can be undercut to facilitate retention of the lead lines 340 in the second portion 311b.

In a further aspect of this embodiment, the first and second portions 311 of the support member 210 include a number of features to reduce stress and strain from use. For example, in one embodiment, the second portion 311b can include generous radiuses 365 extending between the collar 317 and the body of the second portion 311b. The radiuses 365 can reduce strain on the support member 200 from flexing of the cable 230 during use. In another embodiment, the first portion 311a can include an angled surface 367 that bonds to a corresponding surface of the collar 317. The angled joint between the two respective surfaces may provide certain strain relief advantages over a joint that is orientated perpendicular to the cable 230. In addition to the forgoing features, the first portion 311a can also include generous fillet radii between a raised portion 369 that receives the cable 230 and the body of the first portion 311a. In other embodiments, the first and second portions 311a, b can have other strain relief features in addition to those described here, or alternatively, one or more of the features described here may be omitted.

The first and second portions 311 of the support member 210 may be comprised of various flexible and/or elastomeric materials. In one embodiment, for example, both the first portion 311a and the second portion 311b can be manufactured from NUSIL MED-4870 silicone elastomer. In other embodiments, the first and second portions 311 can be manufactured from other flexible materials known to those in the art as being suitable for intracranial implantation for medical applications.

In a further aspect of this embodiment, portions of the lead lines 340 extending away from the support member 210 can be individually housed within inner tubes 342 to insulate the lead lines 340 from each other. The inner tubes 342 can in turn be housed together within an outer tube 344 to form the cable 230 extending between the support member 210 and the connector 233 (FIG. 2). The inner tubes 342 and the outer tube 344 may be comprised of various flexible dielectric materials. For example, in one embodiment, these tubes can be manufactured from a suitable elastomeric material such as NUSIL MED-4765 silicone elastomer. In other embodiments, these tubes can be manufactured from other flexible materials suitable for invasive medical applications and having a wide variety of durometers.

FIG. 3B is a top isometric view of the electrode assembly 200 in a partially assembled state with the support member first portion 311a omitted for purposes of illustration. In one aspect of this embodiment, the first lead line 340a is individually attached to each of the electrodes 220a-c, and the second lead line 340b is individually attached to each of the electrodes 220d-f. In one embodiment, the lead lines 340 can be attached to the electrodes 220 with localized welds 341 applied in the grooves 321. In other embodiments, other methods of attachment can be used. For example, in another embodiment, the lead lines 340 can be brazed to the electrodes 220. In yet another embodiment, portions of the electrodes 220 proximate to the grooves 321 can be coined, crimped, or otherwise deformed to clamp the lead lines 340 into the grooves 321. In another embodiment, the lead lines 340 can be held in the grooves 321 with a suitable adhesive. In a further embodiment, a positive form of attachment can be omitted and the lead lines 340 can be held in the grooves 321 by the first portion 311a (FIG. 3A) when the first portion 311a is bonded to the second portion 311b.

In another aspect of this embodiment, each of the electrodes 220 is installed into a corresponding one of the electrode ports 315. A suitable adhesive, such as NUSIL MED-1511 silicone adhesive, can be applied to portions of the electrodes 220 and/or portions of the second portion 311b (such as the annular recesses 318) during installation to seal and secure the electrodes 220 to the second portion 311b. In this respect, the annular recesses 318 can provide favorable “pocket” to contain the adhesive and position the corresponding electrodes 220. In one embodiment, the adhesive apertures 327 can allow the adhesive to flow through each electrode 220 and extend between the first and second portions 311a, b of the support member 210. This feature can facilitate bonding between the first and second portions 311a, b. Further, this feature can help to secure the electrodes 220 with respect to the support member 210 and prevent an electrode 220 from becoming dislodged by flexing of the support member 210 during implantation of the electrode assembly 200.

In a further aspect of this embodiment, the first lead line 340a is installed into the first groove 313a of the support member second portion 311b, and the second lead line 340b is similarly installed into the second groove 313b. In addition, the cable 230 is inserted through the collar 317 to position a cable end 332 at least approximately between the third electrode 220c and the sixth electrode 220f. By positioning the cable end 332 at this location, bending or flexing of the cable 230 is not likely to cause the support member 210 to fold in a sharp bend along a line 319 proximate to the cable end 332. Instead, the support member 210 is likely to assume a more gentle bend over the region forward of the electrodes 220c, f. Avoiding sharp bending of the support member 210 in this manner may help to limit strains between, for example, the lead lines 340 and the electrodes 220. Such strains can lead to breakage of lead line/electrode connections and possibly result in malfunction of the electrode assembly. Further, sharp bending of the support member 210 may also tend to dislodge an electrode 220 from the support member 210. After the electrodes 220 and the lead lines 340 are installed on the second portion 311b as illustrated in FIG. 3B, the first portion 311a (FIG. 3A) can be bonded to the second portion 311b with a suitable adhesive, such as NUSIL MED-1511 silicone adhesive.

One feature of embodiments of the invention illustrated in FIGS. 2-3B is that in operation the first plurality of electrodes 221 can be biased at a first potential and the second plurality of electrodes 222 can be biased at a second potential. One advantage of this feature is that the group of individual electrodes 220a-c will behave as a single large electrode and the group of electrodes 220d-f will behave as another single large electrode while still providing the overall flexibility of the support member desired for conformance to stimulation sites. In another embodiment, all of the electrodes 220a-f are biased at the same potential to electrically act as a single large electrode. This feature allows an electrical field to be provided over a relatively large area with a flexible substrate. Another feature of embodiments of the invention illustrated in FIGS. 2-3B is the relative thinness of the support member 210 afforded by recessing the lead lines 340 into the electrodes 220. This thinness can help prevent the electrode assembly 200 from applying undue pressure to the patient's cortex at the stimulation site.

Additional features of embodiments of the invention can be seen with reference to FIG. 3B. In this embodiment, the lead lines 340 extend from the cable end 332 to the electrodes 220 (i.e., electrodes 220a, 220d) that are furthest from the cable end 332, and from there the lead lines 340 extend back to the other electrodes on the respective sides of the support member 210. One advantage of this feature is that relative motion of the lead lines 340 caused by, for example, movement of the cable 230 may be attenuated or dampened before the lead lines reach the electrodes 220. Dampening this motion can reduce strain between the lead lines 340 and the electrodes 220. Further, alignment of the grooves 321 in the electrodes 220 with the grooves 313 in the support member second portion 311b can also reduce strain between the lead lines 340 and the electrodes 220. All of the foregoing features may enhance the functionality and/or durability of the electrode assembly 200, thereby reducing the risk of damage that could render the electrode assembly 200 inoperative.

FIG. 4 is a top isometric view of a partially assembled electrode assembly 400 configured in accordance with another embodiment of the invention. The electrode assembly 400 is at least generally similar in structure and function to the electrode assembly 200 described above with reference to FIGS. 2-3B. In one aspect of this embodiment, however, the electrode assembly 400 includes a third lead line 440a and a fourth lead line 440b. The third lead line 440a extends through the first grooves 321a of the first plurality of electrodes 221. Similarly, the fourth lead line 440b extends through the first grooves 321a of the second plurality of electrodes 222. In another aspect of this embodiment, the first lead line 340a is installed in the third groove 313c of the support member second portion 311b instead of the first groove 313a. From the third groove 313c, the first lead line 340a extends into the second groove 321b of the second electrode 220b to intersect the third lead line 440a. Similarly, the second lead line 340b is installed in the fourth groove 313d of the support member second portion 311b instead of the second groove 313b. From the fourth groove 313d, the second lead line 340b extends into the second groove 321b of the fifth electrode 220e to intersect the fourth lead line 440b.

The lead lines 340, 440 of this embodiment can be attached to the electrodes 220 in a number of different ways. For example, referring to the first plurality of electrodes 221, in one embodiment, the third lead line 440a can be attached to the second electrode 220b with welds 441a, b positioned on opposite sides of the first lead line 340a. The first lead line 340a can be attached to the second electrode 220b with a similar weld 441c. The third lead line 440a can be attached to the first and, third electrodes 220a, c with welds 341 as shown above in FIG. 3B. The foregoing method of attaching the lead lines 340, 440 to the first plurality of electrodes 221 are equally applicable to the second plurality of electrodes 222. In other embodiments, other methods can be used to attach the lead lines 340, 440 to the electrodes 220. For example, in one other embodiment, the electrodes 220 can be coined as described above to attach the lead lines 340, 440 to the electrodes 220.

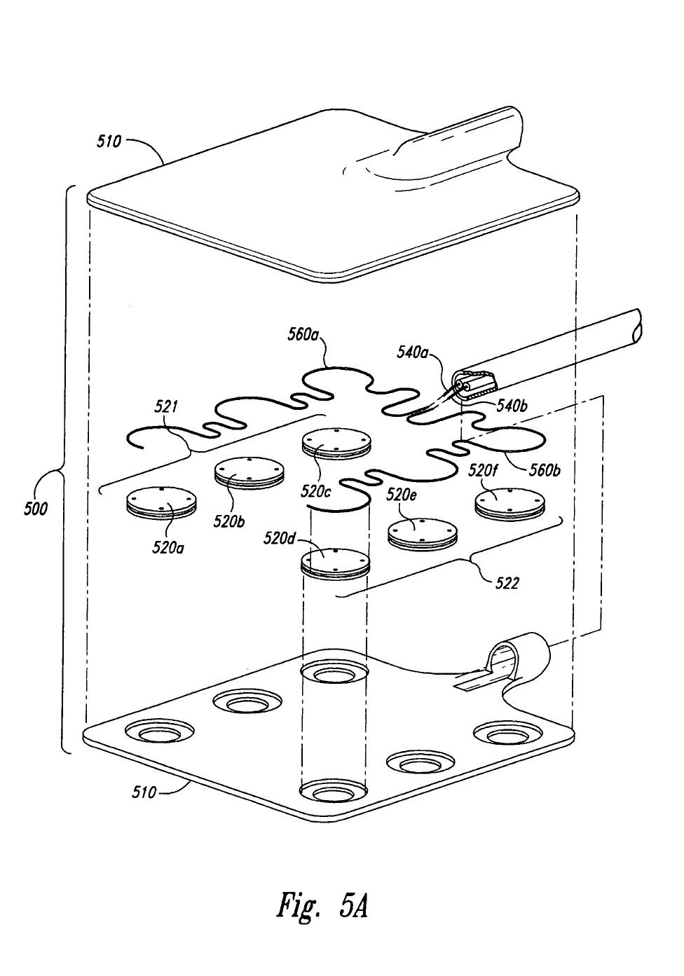

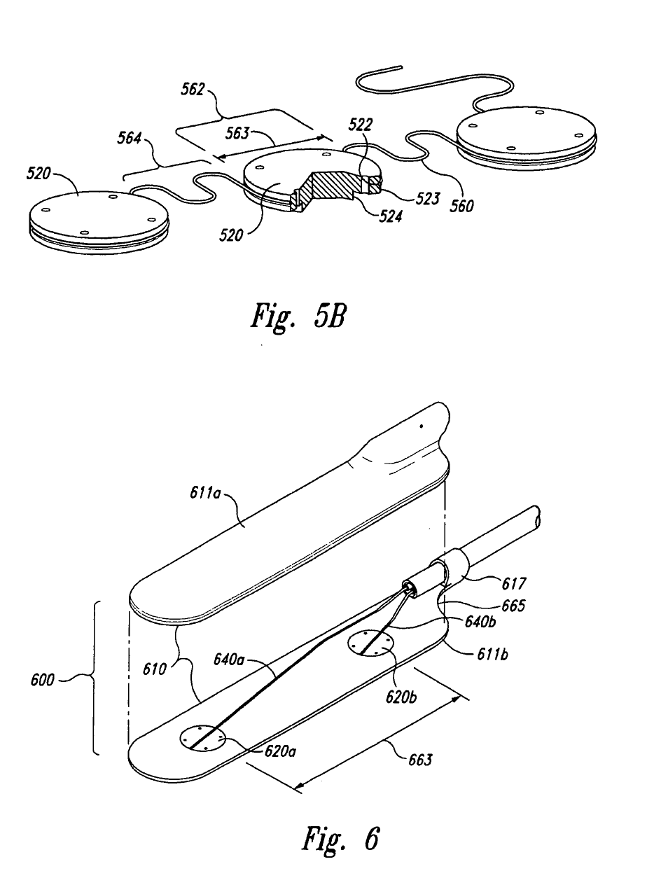

FIG. 5A is an exploded isometric view of an implantable electrode assembly 500 configured in accordance with another embodiment of the invention. FIG. 5B is an enlarged, partial cutaway isometric view of a plurality of interconnected electrodes 520 from the electrode assembly 500 of FIG. 5A. Referring first to FIG. 5A, in one aspect of this embodiment, the electrode assembly 500 includes a flexible support member 510 that is at least generally similar in structure and function to the support member 210 described above with reference to FIGS. 2-4. In another aspect of this embodiment, however, the electrode assembly 500 further includes a first preformed wire 560a interconnecting a first plurality of electrodes 521 (illustrated as electrodes 520a-c), and a second preformed wire 560b interconnecting a second plurality of electrodes 522 (illustrated as electrodes 520d-f). The preformed wires 560a, b can be welded, soldered, crimped, or otherwise connected to lead lines 540a, b. In operation, the first plurality of electrodes 521 can be biased at a first potential and the second plurality of electrodes 522 can be biased at a second potential to generate an electric field between the electrodes for stimulation of a site.

Referring next to FIG. 5B, in a further aspect of this embodiment, each of the electrodes 520 can include an annular groove 522 extending circumferentially around a first cylindrical portion 523. In addition, each of the preformed wires 560 can include a plurality of retaining portions 562 spaced apart by flex portions 564. The retaining portions 562 are shaped and sized to extend at least partially around the electrodes 520 and fit into the grooves 522 to interconnect the electrodes 520 together. In one embodiment, each retaining portion 562 has an opening dimension 563 that is smaller than the diameter of the corresponding electrode 520. As a result, the electrode 520 will be “captured” in the retaining portion 562 when the preformed wire 560 snaps into place in the groove 522. In addition to relying on spring force, the preformed wires 560 can also be attached to the electrodes 520 in a number of different ways. For example, in one embodiment, the electrodes 520 can be coined or otherwise deformed proximate to the groove 522 to clamp the preformed wires 560 in place. In another embodiment, the preformed wires 560 can be welded to the electrodes 520.

In yet another aspect of this embodiment, the flex portions 564 can be configured to allow for relative motion between the electrodes 520 while maintaining the connection between the electrodes 520. In the illustrated embodiment, for example, the flex portions 564 include one or more convolutions. In other embodiments, the flex portions 564 can have other configurations to accommodate relative motion between the electrodes 520.

The preformed wires 560 may be comprised of various conductive materials. For example, in one embodiment, the preformed wires 560 can include MP35N wire having a diameter of about 0.127 mm. In another embodiment, the preformed wires 560 can include quadrifiler coil having a diameter of 0.254 mm. In a further embodiment, the preformed wires 560 can include other conductive metals such as various steels, nickel, platinum, titanium, and/or gold.

Although the preformed wires 560 of the illustrated embodiment are resilient wires, in other embodiments, nonpreformed and/or nonresilient wires can be used to interconnect the electrodes 520 by attaching to the sides of the electrodes 520. For example, in one other embodiment, the electrodes 520 can be interconnected by a single strand of nonresilient wire that is welded into a small portion of each groove 522 without wrapping very far around the electrode 520. In another embodiment, the electrodes 520 can be interconnected by a coiled wire that is similarly welded into the grooves 522. In all of these embodiments, the annular grooves 522 should be appropriately sized to accommodate the particular type of wire used. In yet other embodiments, the grooves 522 can be omitted and the interconnecting wires can be welded directly to the sides of the electrodes 520. It will be appreciated that one benefit of these embodiments is that the interconnecting wires (e.g., the preformed wires 560) can interconnect the electrodes 520 without extending over the tops of the electrodes 520, thereby keeping the thickness of the support member to a minimum.

FIG. 6 is a partially exploded top isometric view of an electrode assembly 600 having a 2×1 electrode array configured in accordance with another embodiment of the invention. In one aspect of this embodiment, the electrode assembly 600 includes a first electrode 620a connected to a first lead line 640a, and a second electrode 620b connected to a second lead line 640b. The electrodes 620 are carried by a flexible support member 610 having a first portion 611a and a second portion 611b. The support member 610, the lead lines 640, and the electrodes 620 can be at least generally similar in structure and function to the analogous structures described above with reference to FIGS. 2-5. The 2×1 electrode array of the electrode assembly 600 may have certain advantages, however, over larger arrays in some applications where, for example, the stimulation site is relatively small.

In another aspect of this embodiment, the first and second electrodes 620a, b can be spaced apart by a distance 662. In one embodiment, the distance 662 can be greater than about 31 mm, such as about 35 mm, to provide or induce a desired therapeutic effect that may be enhanced by such spacing. In other embodiments, the distance 662 can be less than about 31 mm and/or determined in accordance with certain anatomical considerations and/or the nature or extent of the patient's disorder or condition.

In a further aspect of this embodiment, the second portion 611b includes a collar 617 that is at least partially offset toward one side of the second portion 611b. One advantage of this feature is that it allows each of the first and second lead lines 640a, b to have an at least generally direct path to the corresponding electrode 620a, b, respectively. Here, an “at least generally direct path,” means that the lead line 640a, for example, does not have to cross over, or make a substantial detour around, the second electrode 620b to get to the first electrode 620a. In addition, the second portion 611b can include a generous radius 665 between the collar 617 and the body of the second portion 611b. The radius 665 can favorably reduce strain caused by flexing of the collar 617. In other embodiments, however, the collar 617 may be generally centered relative to the second portion 611b, and/or the radius 665 my be reduced or omitted.

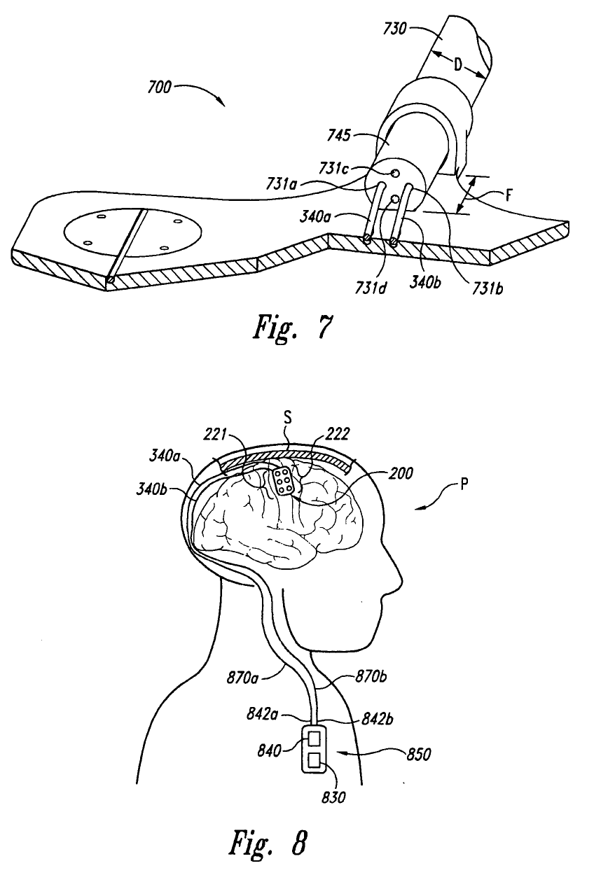

FIG. 7 is an enlarged cutaway isometric view of a portion of an electrode assembly 700 having a cable 730 configured in accordance with another embodiment of the invention. In one aspect of this embodiment, the cable 730 includes a flexible multi-lumen tube 745 having a plurality of passages 731 (shown as a first passage 731a, a second passage 731b, a third passage 731c, and a fourth passage 731d). In the illustrated embodiment, the first lead line 340a extends through the first passage 731a, and the second lead line 340b extends through the opposing second passage 731b. This passage arrangement leaves the third passage 731c and the opposing fourth passage 731d open. The open third and fourth passages 731c, d may enhance flexibility of the multi-lumen tube 745 by giving tube material room to move as the multi-lumen tube 745 is flexed. In other embodiments, however, a cable in accordance with the invention can include a multi-lumen tube having all of its passages occupied by lead lines such that none of the passages are left open. Further, although the illustrated embodiment includes four individual passages 731a-d, in other embodiments, multi-lumen tubes having more or fewer passages can be used depending on factors such as the number of lead lines to accommodate.

In another aspect of this embodiment, the passages 731 may be filled with adhesive for a distance F proximate to each end of the multi-lumen tube 745. This adhesive can prevent or reduce relative motion between the lead lines 340 and the multi-lumen tube 745 as the multi-lumen tube 745 is flexed or stretched during use. Reducing this relative motion may reduce internal abrasion of the multi-lumen tube 745 and/or strain of the lead lines 340 that could result in malfunction of the electrode assembly 700.

One advantage of the cable 730 over the cable 230 described above (FIGS. 2-3B) is the smaller diameter of the multi-lumen tube 745. For example, in one embodiment, the cable 230 can have a diameter of about 2 mm and the cable 730 can have a diameter of about 1.6 mm. As those of ordinary skill in the relevant art will appreciate, a smaller diameter can facilitate easier insertion of the cable 730 through, for example, a subclavicular tunnel. A further advantage of the cable 730 is that additional inner tubes are not required to insulate the lead lines 340 from each other.

FIG. 8 is a side view illustrating a system for applying electrical stimulation to a site on a patient P in accordance with an embodiment of the invention. In the illustrated embodiment, the stimulation site is located at or near the surface of the cortex of the patient P. In other embodiments, the system, or various aspects thereof, can be used to apply electrical stimulation to other sites on the patient P. In one aspect of this embodiment, the stimulation system includes a stimulus unit 850 and the electrode assembly 200. Although the electrode assembly 200 is used here for purposes of illustration, in other embodiments, the stimulation system can include other electrode assemblies in accordance with the invention.

In another aspect of this embodiment, the stimulus unit 850 generates and outputs stimulus signals, such as electrical and/or magnetic stimuli. In the illustrated embodiment, the stimulus unit 850 is generally an implantable pulse generator that is implanted into the patient P in a thoracic, abdominal, or subclavicular location. In other embodiments, the stimulus unit 850 can be an IPG implanted in the skull or just under the scalp of the patient P. For example, in one other embodiment, the stimulus unit 850 can be implanted above the neck-line or in the skull of the patient P as set forth in U.S. patent application Ser. No. 09/802,808.

In a further aspect of this embodiment, the stimulus unit 850 includes a controller 830 and a pulse system 840. The controller 830 can include a processor, a memory, and computer-readable instructions stored on a programmable computer-readable medium. The controller 830 can be implemented as a computer or a microcontroller. The programmable medium can include software loaded into the memory and/or hardware that performs, directs, and/or facilitates neural stimulation procedures.

In yet another aspect of this embodiment, the pulse system 840 can generate energy pulses that are outputted to a first terminal 842a and a second terminal 842b. The first terminal 842a can be biased at a first potential and the second terminal can be biased at a second potential at any given time. In one embodiment, the first potential can have a first polarity and the second potential can have a second polarity or be neutral. That is, the first potential can be either anodal or cathodal, and the second potential can be opposite the first polarity or neutral. In another embodiment, the first potential and the second potential can have the same polarity.

In a further aspect of this embodiment, the electrical stimulation system does not include an intermediate connector between the electrode assembly 200 and the stimulus unit 850. One advantage of this feature is that it provides a complete end-to-end system without the bulk of an intermediate connector and the associated risk of connector failure. In other embodiments, however, one or more connectors can be included between the electrode assembly 200 and the stimulus unit 850. In one such other embodiment, the first and second terminals 842a, b can be included in a single connector connecting the electrode assembly 200 to the pulse system 840.

As described in detail above with reference to FIGS. 2-3B, the electrode assembly 200 includes the first plurality of electrodes 221 and the second plurality of electrodes 222 carried by the support member 210. In the illustrated embodiment, the support member 210 is implanted under the skull S of the patient P so that the electrodes 220 contact a stimulation site on, or at least proximate to, the surface of the cortex of the patient. As also described above, the first plurality of electrodes 221 are connected to the first lead line 340a, and the second plurality of electrodes 222 are connected to the second lead line 340b. The first lead line 340a can be coupled to a first link 870a to electrically connect the first plurality of electrodes 221 to the first terminal 842a of the pulse system 840. The second lead line 340b can be similarly coupled to a second link 870b to connect the second plurality of electrodes 222 to the second terminal 842b of the pulse system 840. The links 870 can be wired or wireless links. In the illustrated embodiment, the pulse system 840 biases the first plurality of electrodes 221 at the first polarity and the second plurality of electrodes 222 at the second polarity. Such biasing can induce an electrical pulse between the first plurality of electrodes 221 and the second plurality of electrodes 222 to provide bipolar stimulation.

In another embodiment, all of the electrodes 220 can be biased at the same potential in an isopolar arrangement. In this embodiment, the electrode assembly 200 can generate an electrical pulse between the electrodes 220 and a separate pole (not shown in FIG. 8) implanted in the body of the patient P. Alternatively, the electrical pulse can be generated between the electrodes 220 and a portion of the patient's body, a housing of the stimulus unit 850, and/or another point.

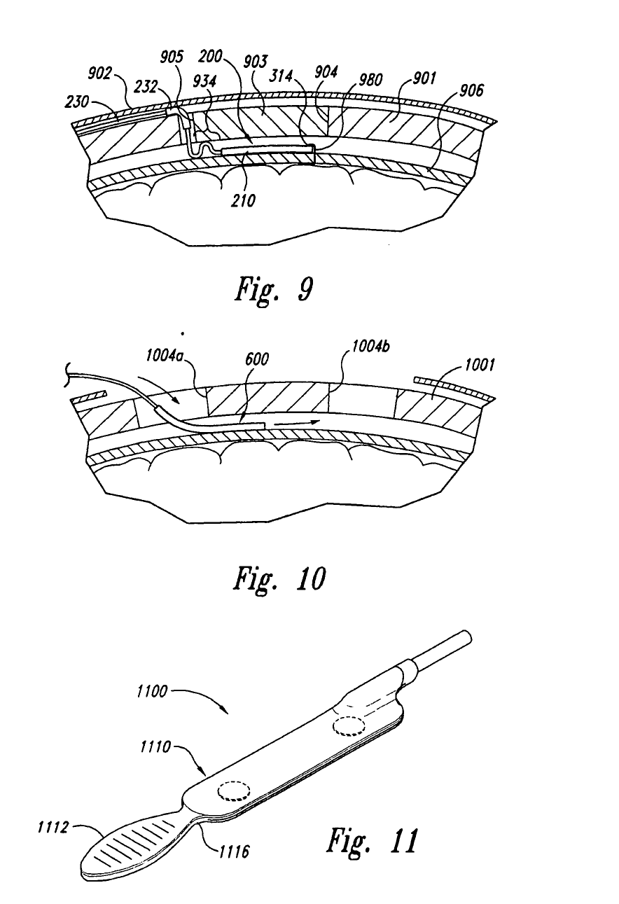

FIG. 9 is an enlarged cross-sectional view of the electrode assembly 200 implanted at a stimulation site on a patient in accordance with an embodiment of the invention. In one aspect of this embodiment, the electrode assembly 200 is implanted into the patient by forming an opening in the scalp 902 and removing a skull portion 903 to form a hole 904 through the skull 901. Further, a notch 905 can be cut in the skull portion 903 to accommodate the cable 230. The hole 904 should be sized to receive the electrode assembly 200; however, in some applications the hole 904 can be smaller than the electrode assembly 200 due to the flexibility of the support member 210.

In another aspect of this embodiment, the support member 210 can be stitched or otherwise attached to the dura mater 906 at the stimulation site by looping one or more couplings 980 through the dura mater 906 and through one or more of the coupling apertures 314 in the support member 210. In one embodiment, the coupling 980 can include a simple suture. In other embodiments, other forms of attachment can be used to at least temporarily hold the support member 210 in position at the stimulation site. For example, in one other embodiment, the coupling apertures 314 can be omitted and a needle can be used to extend sutures or other couplings through the support member material. A bio-compatible adhesive can also be used in conjunction with, or as an alternative to, the sutures. In yet another embodiment, a positive form of attachment between the support member 210 and the dura mater 906 can be omitted. After implantation of the electrode assembly 200 at the stimulation site, the skull portion 903 is replaced and sutured and/or otherwise attached to the skull 901 to at least partially cover the hole 904.

In a further aspect of this embodiment, the cable 230 can include a preformed convoluted portion 934 proximate to the junction between the cable 230 and the support member 210. The convoluted portion 934 can act as a strain relief that prevents the support member 210 from exerting undue pressure on the stimulation site as a result of excessive cord movement. For example, if a practitioner momentarily pushes on the cable 230 during implantation of the electrode assembly 200, or if the cable 230 shifts for another reason after implantation, the convoluted portion 934 may act to dampen this motion and avoid transmitting it to the support member 210. Otherwise, such motion of the support member 210 may apply undesirable pressure to the stimulation site, resulting in discomfort to the patient. In yet another aspect of this embodiment, the sleeve 232 may protect the cable 230 from abrasion on the edge of the notch 905.

FIG. 10 is an enlarged, cross-sectional side view of the electrode assembly 600 of FIG. 6 being installed at a stimulation site in accordance with an embodiment of the invention. In one aspect of this embodiment, a first hole 1004a and a second hole 1004b are formed relatively close to each other in the skull 1001. In one embodiment, for example, the holes 1004 can be spaced apart by a distance of about 15 mm to about 35 mm. A practitioner inserts the electrode assembly 600 through the first hole 1004a to position the electrode assembly 600 between the skull 1001 and a stimulation site. The practitioner may then access the electrode assembly 600 from the second hole 1004b and pull on the electrode assembly 600 to finish positioning it at the stimulation site between the first hole 1004a and the second hole 1004b.

FIG. 11 is a top, partially hidden isometric view of an electrode assembly 1100 configured in accordance with another embodiment of the invention. In one aspect of this embodiment, the electrode assembly 1100 is at least generally similar in structure and function to the electrode assembly 600 described above with reference to FIG. 6. In another aspect of this embodiment, however, the electrode assembly 1100 includes a positioning portion 1112 extending from a forward portion of a support member 1110. With reference to FIG. 10, the positioning portion 1112 can facilitate positioning of the electrode assembly 1100 underneath the patient's skull by providing a portion of the support member 1110 that a practitioner can pull on without fear of damaging the electrode array. In one embodiment, the positioning portion 1112 can be integrally molded as part of the support member 1110, and can include a necked-down region 1116. After the practitioner has sufficiently positioned the electrode assembly 1100 at a stimulation site, the practitioner can remove the positioning portion 1112 by cutting through the necked-down region 1116.

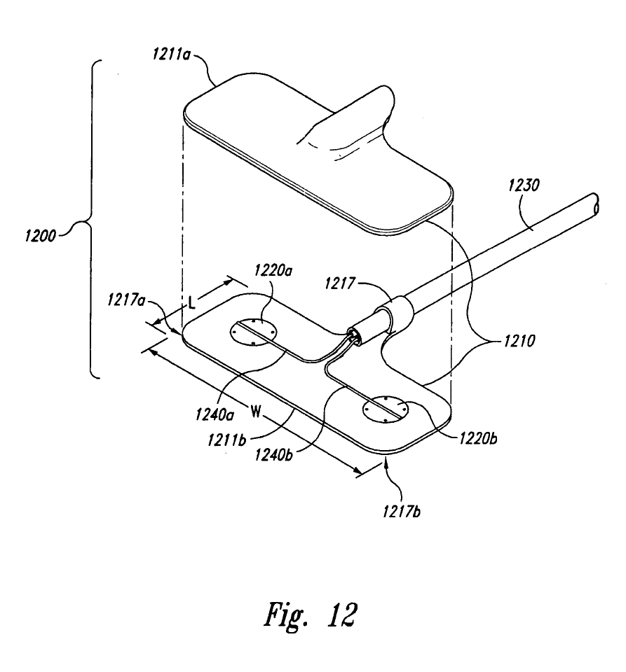

FIG. 12 is a partially exploded top isometric view of an electrode assembly 1200 configured in accordance with another embodiment of the invention. In one aspect of this embodiment, the electrode assembly 1200 includes an electrode array comprising a first electrode 1220a spaced apart from a second electrode 1220b. The electrodes 1220 can be carried by a flexible support member 1210 having a first portion 1211a and a second portion 1211b. The first electrode 1220a can be connected to a first lead line 1240a, and the second electrode 1220b can be connected to a second lead line 1240b. The lead lines 1240 can be housed in a cable 1230 that is received in a collar 1217 formed in the second portion 1211b of the support member 1210.

In another aspect of this embodiment, the support member 1210 includes a first end 1217a spaced apart from a second end 1217b defining a width W therebetween. The support member 1210 can further include a length L that is transverse to the width W and less than the width W. In a further aspect of this embodiment, the cable 1230 can be attached to the second portion 1211b of the support member 1210 at least generally between the first end 1217a and the second end 1217b. This support member configuration may provide a favorable orientation of the electrodes 1220 at certain stimulation sites to provide or induce a desired therapeutic effect.

Although the support member 1210 of the illustrated embodiment is at least generally rectangular, in other embodiments, the support member 1210 can have other shapes wherein the width W exceeds the length L and the cable 1230 is attached to the support member between the first and second ends. For example, in one such embodiment, the support member can be at least generally oval in shape.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number, respectively. Additionally, the words “herein,” “above” and “below” and words of similar import, when used in this application, shall refer to this application as a whole and not to any particular portions of this application.

The description of embodiments of the invention is not intended to be exhaustive or to limit the invention to the precise forms disclosed. While specific embodiments of, and examples for, the invention are described herein for illustrative purposes, other embodiments are possible within the scope of the invention, as those skilled in the relevant art will recognize. For example, while certain embodiments have been described in the context of intracranial therapy, it is expected that other embodiments may be useful in other applications, such as spinal cord therapy. Further, aspects of the invention can be modified, if necessary, to employ the systems, functions and concepts of the patent applications cited above that are incorporated herein by reference. These and other changes can be made to the invention in light of the detailed description.

From the foregoing, it will be appreciated that specific embodiments of the invention have been described herein for purposes of illustration, but that various modifications may be made without deviating from the spirit and scope of the invention. Accordingly, the invention is not limited except as by the appended claims.

1. An implantable electrode assembly comprising:
a flexible support member;
a first plurality of electrodes carried by the support member;
a second plurality of electrodes carried by the support member and spaced apart from the first plurality of electrodes;
a first lead at least partially carried by the support member and electrically interconnecting the first plurality of electrodes; and
a second lead at least partially carried by the support member and insulated from the first lead, the second lead electrically interconnecting the second plurality of electrodes.

2. The implantable electrode assembly of claim 1 wherein the first lead is configured to be connected to a stimulus unit for biasing the first plurality of electrodes at a first potential, wherein the second lead is configured to be connected to the stimulus unit for biasing the second plurality of electrodes at a second potential, and wherein biasing the first plurality of electrodes at the first potential and the second plurality of electrodes at the second potential with the stimulus unit generates an electrical field between the first and second pluralities of electrodes when the support member is placed at a stimulation site.

3. The implantable electrode assembly of claim 1 wherein the support member is at least generally rectangular having a first side edge spaced apart from an opposite second side edge, wherein the first plurality of electrodes are at least generally aligned in a first row proximate to the first side edge, and wherein the second plurality of electrodes are at least generally aligned in a second row proximate to the second side edge.

4. The implantable electrode assembly of claim 1 wherein at least one of the first plurality of electrodes has a groove, and wherein the first lead is at least partially disposed in the groove.

5. The implantable electrode assembly of claim 1 wherein at least one of the first plurality of electrodes has a flat surface with a groove, and wherein the first lead is at least partially disposed in the groove.

6. The implantable electrode assembly of claim 1 wherein at least one of the first plurality of electrodes has a cylindrical surface with a groove, and wherein the first lead is at least partially disposed in the groove.

7. The implantable electrode assembly of claim 1 wherein the support member includes a first portion bonded to a complimentary second portion, wherein the second portion includes at least a first preformed groove facing the first portion, and wherein the first lead is at least partially disposed in the first preformed groove.

8. The implantable electrode assembly of claim 1 wherein at least one of the first plurality of electrodes includes a first electrode groove, wherein the support member includes at least a first support member groove, wherein at least a portion of the first support member groove is aligned with the first electrode groove, and wherein the first lead is at least partially disposed in the first support member groove and the first electrode groove.

9. An implantable electrode assembly comprising:
a flexible support member;
a first electrode carried by the support member;
at least a second electrode spaced apart from the first electrode and carried by the support member; and
a lead electrically connecting the first electrode to the second electrode.

10. The implantable electrode assembly of claim 9 wherein the lead is a first lead, and further comprising:
at least a third electrode carried by the support member; and
a second lead electrically insulated from the first lead and electrically connected to the third electrode.

11. The implantable electrode assembly of claim 10 wherein the first lead is configured to be connected to a first terminal for biasing of the first and second electrodes at a first potential, wherein the second lead is configured to be connected to a second terminal for biasing of the third electrode at a second potential, and wherein biasing of the first and second electrodes at the first potential and the third electrode at the second potential generates an electrical field when the support member is placed at a stimulation site.

12. The implantable electrode assembly of claim 9, further comprising a cable extending outwardly from the support member, the cable including a tube at least partially housing the lead, the cable further including a cable end received by the support member, the first electrode being positioned a first distance from the cable end, the second electrode being positioned a second distance from the cable end, the second distance being less than the first distance, and wherein a portion of the lead extends from the cable end to the first electrode and then from the first electrode to the second electrode.

13. An implantable electrode assembly comprising:
a flexible support member;
at least one electrode carried by the support member, the electrode having a surface with a groove; and
an electrical lead at least partially disposed in the groove, the lead configured to connect the electrode to a stimulus unit for biasing of the electrode at an electrical potential.

14. The implantable electrode assembly of claim 13 wherein the surface of the electrode with the groove is at least generally flat.

15. The implantable electrode assembly of claim 13 wherein the surface of the electrode with the groove is at least generally curved.

16. The implantable electrode assembly of claim 13 wherein the groove is an annular groove extending around the electrode.

17. The implantable electrode assembly of claim 13 wherein the groove is a first groove and the lead is a first lead, wherein the electrode further includes a second groove, and wherein the electrode assembly further includes a second lead at least partially disposed in the second groove.

18. The implantable electrode assembly of claim 13 wherein the lead includes a plurality of metallic strands.

19. The implantable electrode assembly of claim 13 wherein the lead includes at least one strand of MP35N wire.

20. The implantable electrode assembly of claim 13 wherein the groove is a circumferential groove extending around the electrode, and wherein the lead includes a preformed resilient wire configured to fit into the groove and extend at least partially around the electrode.

21. The implantable electrode assembly of claim 13 wherein the lead is welded to the electrode.

22. The implantable electrode assembly of claim 13 wherein the lead is held in the groove by deformation of the electrode at least proximate to the groove.

23. The implantable electrode assembly of claim 13 wherein the lead is held in the groove by adhesive.

24. The implantable electrode assembly of claim 13 wherein the electrode includes at least one of platinum and iridium.

25. The implantable electrode assembly of claim 13 wherein the lead includes at least one of nickel and cobalt.

26. The implantable electrode assembly of claim 13 wherein the support member includes at least one preformed groove, and wherein the lead is at least partially disposed in the preformed groove.

27. The implantable electrode assembly of claim 13 wherein the support member includes a first portion bonded to a complimentary second portion, wherein the second portion includes at least one preformed groove facing the first portion, and wherein the lead is at least partially disposed in the preformed groove of the second portion.

28. The implantable electrode assembly of claim 13 wherein the support member includes at least one preformed groove, wherein at least a portion of the preformed groove in the support member is aligned with the groove in the electrode, and wherein the lead is at least partially disposed in the preformed groove of the support member.

29. The implantable electrode assembly of claim 13 wherein the electrode is a first electrode and the groove is a first groove, wherein the electrode assembly further comprises a second electrode having a second groove, and wherein the lead is at least partially disposed in the second groove.

30. The implantable electrode assembly of claim 13 wherein the electrode is a first electrode, and further comprising:
a second electrode offset from the first electrode; and
a cable extending outwardly from the support member, the cable including a tube at least partially housing the lead, the cable further including a cable end received by the support member, the first electrode being positioned a first distance from the cable end, the second electrode being positioned a second distance from the cable end, the second distance being less than the first distance, and wherein a portion of the lead extends from the cable end to the first electrode and then from the first electrode to the second electrode.

31. The implantable electrode assembly of claim 13 wherein the electrode is a first electrode, and further comprising:
a second electrode spaced apart from the first electrode to define a space therebetween; and
a cable extending outwardly from the support member, the cable including a tube at least partially housing the lead, the cable further including a cable end received by the support member, the cable end being positioned in the space between the first electrode and the second electrode.

32. An implantable electrode assembly comprising:
a flexible support member;
at least one electrode carried by the support member, the electrode having a first surface positioned to contact a portion of a patient and a second surface positioned opposite to the first surface; and
a lead contacting the electrode at least generally between the first surface and the second surface.

33. The implantable electrode assembly of claim 32 wherein the first and second surfaces define two offset parallel planes.

34. The implantable electrode assembly of claim 32 wherein the electrode further includes at least a first groove formed adjacent to the second surface, and wherein the lead is at least partially disposed in the groove.

35. The implantable electrode assembly of claim 32 wherein the electrode further includes a third surface extending at least partially between the first and second surfaces, wherein the electrode still further includes a groove formed in the third surface, and wherein the lead is at least partially disposed in the groove.

36. The implantable electrode assembly of claim 32 wherein the electrode further includes a cylindrical surface extending at least partially between the first and second surfaces, wherein the electrode still further includes a groove formed in the cylindrical surface, and wherein the lead is at least partially disposed in the groove.

37. The implantable electrode assembly of claim 32 wherein the electrode further includes at least one aperture, and wherein the lead is at least partially disposed in the aperture.

38. The implantable electrode assembly of claim 32 wherein the first and second surfaces define an electrode thickness of about 1.5 mm.

39. The implantable electrode assembly of claim 32 wherein the first and second surfaces define an electrode thickness of about 1.0 mm.

40. The implantable electrode assembly of claim 32 wherein the first and second surfaces define an electrode thickness of about 0.65 mm.

41. The implantable electrode assembly of claim 32 wherein the electrode further includes first and second cylindrical portions, wherein the first cylindrical portion is positioned adjacent to the first surface and has a first diameter, and wherein the second cylindrical portion is positioned adjacent to the second surface and has a second diameter larger than the first diameter.

42. The implantable electrode assembly of claim 32 wherein the electrode further includes first and second cylindrical portions, wherein the first cylindrical portion is positioned adjacent to the first surface and has a first diameter, wherein the second cylindrical portion is positioned adjacent to the second surface and has a second diameter larger than the first diameter, and wherein the electrode still further includes a groove formed in the second portion of the electrode, the lead being at least partially disposed in the groove.

43. The implantable electrode assembly of claim 32 wherein the electrode further includes first and second cylindrical portions, wherein the first cylindrical portion is positioned adjacent to the first surface and has a first diameter, wherein the second cylindrical portion is positioned adjacent to the second surface and has a second diameter larger than the first diameter, and wherein the electrode still further includes a groove formed in the second portion of the electrode adjacent to the second surface, the lead being at least partially disposed in the groove.

44. An implantable electrode assembly comprising:
a flexible support member;
a first electrode carried by the support member, the first electrode having a first surface positioned to contact a portion of a patient and a second surface positioned opposite to the first surface;
a second electrode carried by the support member, the second electrode having a third surface positioned to contact a portion of the patient and a fourth surface positioned opposite to the third surface; and
an electrical lead at least partially carried by the support member, the lead contacting the first electrode at a first location positioned at least generally between the first surface and the second surface, the lead further contacting the second electrode at a second location positioned least generally between the third surface and the fourth surface.

45. The implantable electrode assembly of claim 44 wherein the first electrode further includes a first groove, wherein the second electrode further includes a second groove, and wherein the lead is at least partially disposed in the first and second grooves.

46. The implantable electrode assembly of claim 44 wherein the lead is a first lead, and further comprising:
at least a third electrode carried by the support member; and
a second electrical lead carried by the support member and insulated from the first lead, the second lead contacting the third electrode.

47. The implantable electrode assembly of claim 44 wherein the lead is a first lead, and further comprising:
at least a third electrode carried by the support member; and
a second electrical lead carried by the support member and insulated from the first lead, the second lead contacting the third electrode, wherein the first lead is configured to bias the first and second electrodes at a first potential, and wherein the second lead is configured to bias at least the third electrode at a second potential to generate an electric field between the first and second electrodes and the third electrode.

48. An implantable electrode assembly comprising:
a flexible support member;
a first electrode carried by the support member;
at least a second electrode carried by the support member;
an electrical lead carried by the support member, the lead contacting the first electrode and the second electrode; and
a cable extending outwardly from the support member, the cable including a tube at least partially housing the lead, the cable further including
a cable end at least partially received by the support member, wherein the first electrode is positioned a first distance from the cable end and the second electrode is positioned a second distance from the cable end, the second distance being less than the first distance, and wherein the lead extends from the cable end to the first electrode and then from the first electrode to the second electrode.

49. The implantable electrode assembly of claim 48 wherein the lead is a first lead, and further comprising:
a third electrode carried by the support member;
at least a fourth electrode carried by the support member; and
a second electrical lead carried by the support member and insulated from the first lead, the second lead contacting the third electrode and the fourth electrode, wherein the third electrode is positioned a third distance from the cable end and the fourth electrode is positioned a fourth distance from the cable end, the fourth distance being less than the third distance, and wherein the second lead extends from the cable end to the third electrode and then from the third electrode to the fourth electrode.

50. An implantable electrode assembly comprising:
a flexible support member having a first end spaced apart from a second end defining a width therebetween, the support member further having a length transverse to the width, the length being less than the width;
a first electrode carried by the support member;
at least a second electrode carried by the support member and spaced apart from the first electrode; and
at least a first lead carried by the support member and electrically connected to at least the first electrode, wherein the first lead is at least partially housed in cable attached to the support member between the first and second ends.

51. The implantable electrode assembly of claim 50 wherein the support member is at least generally rectangular, and wherein the first electrode is positioned at least proximate to the first end of the support member and the second electrode is positioned at least proximate to the second end of the electrode.

52. The implantable electrode assembly of claim 50 wherein the support member is at least generally rectangular and the cable is attached to the support member at least generally mid-way between the first end and the second end.

53. The implantable electrode assembly of claim 50, further comprising a second lead at least partially carried by the support member and electrically connected to the second electrode, wherein the second lead is at least partially housed in the cable.

54. The implantable electrode assembly of claim 50 wherein the support member includes a first portion bonded to a complimentary second portion, wherein the second portion includes at least a first preformed groove facing the first portion, and wherein the first lead is at least partially disposed in the first preformed groove.

55. A system for applying electrical stimulation at a site proximate to a surface of the cortex of a patient, the system comprising:
a stimulus unit having a pulse system including a first terminal that can be biased at a first potential and a second terminal that can be biased at a second potential; and
an implantable electrode assembly having:
a flexible support member;
a first electrode carried by the support member;
at least a second electrode spaced apart from the first electrode and carried by the support member; and
a lead electrically connecting the first electrode to the second electrode, wherein the lead is configured to be connected to the first terminal for biasing of the first and second electrodes at the first potential.

56. The electrical stimulation system of claim 55 wherein the lead is a first lead, and further comprising:
at least a third electrode carried by the support member; and
a second lead electrically insulated from the first lead and electrically connected to the third electrode, wherein the second lead is configured to be connected to the second terminal for biasing of the third electrode at the second potential.

57. The electrical stimulation system of claim 55 wherein the stimulus unit is an implantable unit.

58. The electrical stimulation system of claim 55 wherein the first terminal provides an anodal potential and the second terminal provides a cathodic potential.

59. The electrical stimulation system of claim 55 wherein the stimulus unit is an implantable pulse generator further including a housing and a controller, wherein the pulse system and the controller are carried by the housing.

60. The electrical stimulation system of claim 55 wherein the stimulus unit is an implantable pulse generator configured to be implanted in a human being, and wherein the stimulus unit further includes a controller operatively coupled to the pulse system, the controller including a programmable medium, and wherein the programmable medium contains instructions that cause the pulse system to concurrently electrically bias the first electrode at the first potential and the second electrode at the second potential.

61. A method of manufacturing an implantable electrode assembly, the method comprising:
forming a first portion of a flexible support member;
forming a second portion of the flexible support member, the second portion of the support member configured to carry at least a portion of at least one electrode having a groove;
disposing an electrical lead in the groove of the electrode; and
disposing at least a portion of the electrode in the second portion of the support member.

62. The method of claim 61, further comprising bonding the first portion of the support member to the second portion of the support member.

63. The method of claim 61 wherein the groove in the electrode is a first groove, and further comprising:
disposing at least a portion of the electrical lead in a second groove in the second portion of the support member; and
bonding the first portion of the support member to the second portion of the support member.

64. The method of claim 61, further comprising welding the electrical lead to the electrode.

65. The method of claim 61 wherein the electrical lead includes a preformed resilient wire, and wherein disposing the electrical lead in the groove of the electrode includes extending at least a portion of the lead around a circumference of the electrode.

66. A method of manufacturing an implantable electrode assembly, the method comprising:
forming at least a portion of a flexible support member;
installing a first electrode in the portion of the support member;
installing at least a second electrode in the portion of the support member; and
connecting the first electrode to the second electrode with an electrical lead.

67. The method of claim 66 wherein the portion of the support member is a first portion, and wherein the method further comprises:
forming a second portion of the support member; and
bonding the second portion of the support member to the first portion of the support member, wherein at least a portion of the lead is sandwiched between the first and second portions of the support member.

68. The method of claim 66 wherein the electrical lead is a first lead, and further comprising:
installing at least a third electrode in the portion of the support member; and
connecting a second electrical lead to the third electrode.

69. The method of claim 66 wherein the electrical lead is a first lead, and further comprising:
installing at least a third electrode in the portion of the support member;
connecting a second electrical lead to the third electrode;
housing the first and second electrical leads in a cable tube;
forming a second portion of the support member; and
bonding the second portion of the support member to the first portion of the support member, wherein at least a portion of the first lead and a portion of the second lead are sandwiched between the first and second portions of the support member, and wherein at least a portion of the cable tube spaced apart from the first and second portions of the support member.

70. A method of applying electrical stimulation to a stimulation site on a patient, the method comprising:
positioning a flexible support member at least proximate to the stimulation site, the support member carrying at least a first electrode having a first surface positioned to contact a portion of the stimulation site and a second surface positioned opposite to the first surface, wherein an electrical lead contacts the first electrode at least generally between the first surface and the second surface; and
applying an electrical potential to the lead to bias the first electrode at the first potential.

71. The method of claim 70 wherein the lead is a first lead and the electrical potential is a first electrical potential, and wherein the support member further carries a second electrode having a third surface positioned to contact a portion of the stimulation site and a fourth surface positioned opposite to the third surface, wherein a second electrical lead contacts the second electrode at least generally between the third surface and the fourth surface, and wherein the method further comprises applying a second electrical potential to the second lead to bias the second electrode at the second potential.
